(19) United States
(12) Patent Application Publication
(10) Pub. No.: US 2015/0297313 A1
(43) Pub. Date: Oct. 22, 2015

(54) MARKERLESS TRACKING OF ROBOTIC SURGICAL TOOLS

(71) Applicant: THE TRUSTEES OF COLUMBIA UNIVERSITY IN THE CITY OF NEW YORK, New York, NY (US)

(72) Inventors: Austin Reiter, Great Neck, NY (US); Peter K. Allen, Pleasantville, NY (US)

(73) Assignee: The Trustees of Columbia University in the City of New York, New York, NY (US)

(21) Appl. No.: 14/651,484

(22) PCT Filed: Dec. 13, 2013
(86) PCT No.: PCT/US2013/075014

§ 371 Date: Jun. 11, 2015

Related U.S. Application Data
(60) Provisional application No. 61/737,172, filed on Dec. 14, 2012.

(51) Int. Cl. A61B 19/00; A61B 5/00

(52) U.S. Cl. CPC A61B 19/5244; A61B 19/2203; A61B 5/7267; A61B 2019/5265

(57) ABSTRACT

Appearance learning systems, methods and computer products for three-dimensional markerless tracking of robotic surgical tools. An appearance learning approach is provided that is used to detect and track surgical robotic tools in laparoscopic sequences. By training a robust visual feature descriptor on low-level landmark features, a framework is built for fusing robot kinematics and 3D visual observations to track surgical tools over long periods of time across various types of environments. Three-dimensional tracking is enabled on multiple tools of multiple types with different overall appearances. The presently disclosed subject matter is applicable to surgical robot systems such as the da Vinci® surgical robot in both ex vivo and in vivo environments.

### RELATED APPLICATIONS

This application claims the benefit of U.S. Provisional Application No. 61/737,172, filed Dec. 14, 2012.

### BACKGROUND OF THE DISCLOSED SUBJECT MATTER

1. Field of the Disclosed Subject Matter

Embodiments of the disclosed subject matter relate generally to three-dimensional markerless tracking of robotic medical tools. More particularly, embodiments of the subject matter relate to systems, methods, and computer products for the acquisition and tracking of robotic medical tools through image analysis and machine learning.

2. Description of Related Art

Technological breakthroughs in endoscopy, smart instrumentation, and enhanced video capabilities have allowed for advances in minimally invasive surgery. These achievements have made it possible to reduce the invasiveness of surgical procedures. Computer-aided surgical interventions have been shown to enhance the skills of the physician, and improve patient outcomes. Particularly, robotic hardware and intelligent algorithms have opened the doors to more complex procedures by enhancing the dexterity of the surgeon's movements as well as increasing safety through mechanisms like motion scaling and stereo imaging. To further enhance the surgeon's abilities, robotic surgery systems may include tool tracking functionality to determine the locations of instruments within the surgical field whether within sight of the surgeon or not.

Knowledge of the location and orientation of medial tools in the endoscopic image can enable a wide spectrum of applications. For example, accurate tool localization can be used as a Virtual Ruler capable of measuring the distances between various points in the anatomical scene, such as the sizes of anatomical structures. Graphical overlays can indicate the status of a particular tool, for example, in the case of the firing status of an electro-cautery tool. These indicators can be placed at the tip of the tool in the visualizer which is close to the surgeon's visual center of attention, enhancing the overall safety of using such tools. It can also be useful in managing the tools that are off the screen increasing patient's safety, or for visual serving of motorized cameras.

Tool tracking techniques are generally divided into marker-based systems and marker-less systems. As an example of a markerless tool tracking system, the joints of a robotic surgical system can be equipped with encoders so that the pose of the instruments can be computed through forward kinematics. However, the kinematics chain between the camera and the tool tip can involve on the order of 18 joints over 2 meters. As a result, such approaches are inaccurate, resulting in absolute error on the order of inches.

Previous approaches to marker-based tool tracking have employed specialized fiducial markers to locate the tool in vivo. There are practical challenges to these approaches such as manufacturability and cost. In some approaches, either color or texture is used to mark the tool, and in cases where information about the tool is known a priori, a shape model can be used to confine the search space. One method is to design a custom marker for the surface of the surgical tool, to assist in tool tracking. In one approach, a color marker is designed by analyzing the Hue-Saturation-Value color space to determine what color components aren't common in typical surgical imagery, and the marker is fabricated and placed on a tool to be tracked. A training step creates a kernel classifier which can then label pixels in the frame as either foreground (tool) or background. In some approaches, a marker may comprise three stripes that traverse the known diameter of the tool which allows the estimation of depth information of the tool's shaft from the camera. An alternative example of a marker is a barcode.

Another technique to aid in tracking is to affix assistive devices to the imaging instrument itself. For example, a laser-pointing instrument holder may be used to project laser spots into the laparoscopic imaging frames. This is useful for when the tools move out of the field-of-view of the camera. The laser pattern projected onto the organ surface provides information about the relative orientation of the instrument with respect to the organ. Optical markers are used on the tip of the surgical instruments, and these markers used in conjunction with the image of the projected laser pattern allow for measurements of the pointed organ and the instrument.

Prior approaches to visual feature detection and matching in the computer vision community have applied scale and affine invariant feature descriptors, which have been very successful in matching planar features. However, they work poorly for features on metal surfaces with lighting changes, as in the case of surgical tools with varying poses and light directions. Other prior approaches either result in low accuracy or require the addition of distracting or impractical additional indicia to the surfaces of tools. Thus, there remains a need for an accurate non-invasive tool tracking system that provides knowledge of the locations of tools and enables the use of precise graphical overlays.

### SUMMARY OF THE DISCLOSED SUBJECT MATTER

In one aspect of the disclosed subject matter, a robotic surgical tool tracking method and computer program product is provided. A descriptor of a region of an input image is generated. A trained classifier is applied to the descriptor to generate an output indicative of whether a feature of a surgical tool is present in the region. The location of the feature of the surgical tool is determined based on the output of the trained classifier.

In some embodiments, the descriptor is a covariance descriptor, a scale invariant feature transform descriptor, a histogram-of-orientation gradients descriptor, or a binary robust independent elementary features descriptor. In some embodiments, the trained classifier is a randomized tree classifier, a support vector machine classifier, or an AdaBoost classifier. In some embodiments, the region is selected from within a predetermined area of the input image. In some embodiments, the region is selected from within a mask area indicative of the portion of the input image that corresponds to a tip portion of the surgical tool. In some embodiments wherein the input image contains a plurality of surgical tools, it is determined to which of the plurality of surgical tools the feature corresponds. In some embodiments, the mask area is generated by applying a Gaussian mixture model, image segmentation by color clustering, image segmentation by thresholding, or image segmentation by application of a graph cut algorithm.

In some embodiments where the descriptor is a covariance descriptor, the covariance descriptor comprises an x coordinate, a y coordinate, a hue, a saturation, a color value, a first order image gradient, a second order image gradient, a gradient magnitude, and a gradient orientation. In some embodiments where the classifier is a randomized tree classifier, the randomized tree classifier additionally comprises weights associated with each tree and applying the classifier comprises applying the weights associated with each tree to the outputs of each tree.

It is to be understood that both the foregoing general description and the following detailed description are exemplary and are intended to provide further explanation of the disclosed subject matter claimed.

### BRIEF DESCRIPTION OF THE DRAWINGS

FIG. 1 is a schematic diagram showing the modules of an exemplary embodiment of a system according to the disclosed subject matter.

FIGS. 2A-D depicts sample input and output of the Scene Labeling Module according to embodiments of the disclosed subject matter.

FIGS. 3A-C depict robotic surgical tools according to embodiments of the present disclosure.

FIG. 4 depicts seven naturally-occurring landmarks on a robotic surgical tool in accordance with the system of the present disclosure.

FIG. 5 provides a schematic view of a feature descriptor in accordance with an embodiment of the present subject matter.

FIG. 6 depicts shaft boundary detection output in accordance with an embodiment of the present subject matter.

FIGS. 7A-J depict kinematics output in accordance with an embodiment of the present subject matter.

FIGS. 8A-B depict an evaluation scheme in accordance with the disclosed subject matter.

FIGS. 9A-D depict an example of kinematic latency.

FIGS. 10A-B depict applications of tool tracking in accordance with the present disclosure.

FIG. 11 depicts example appearance changes in a robotic surgical tool typically encountered under different lighting and perspective effects in accordance with the present disclosure.

FIG. 12A shows seven features of a robotic surgical tool that are analyzed in accordance with the present subject matter.

FIG. 12B-H show sample likelihoods on the tip of the robotic surgery tool of FIG. 12A tool overlaid with extrema locations in accordance with the present subject matter.

FIG. 13 is a histogram depicting relative performance of several combinations of descriptors and classifiers according to embodiments of the present disclosure.

### DETAILED DESCRIPTION OF EXEMPLARY EMBODIMENTS

Reference will now be made in detail to exemplary embodiments of the disclosed subject matter, examples of which are illustrated in the accompanying drawing. The method and corresponding steps of the disclosed subject matter will be described in conjunction with the detailed description of the system.

Generally, the subject matter described herein provides a system, method, and computer product for tracking robotic surgical tools in vivo or ex vivo via image analysis that provides a level of accuracy not available in existing systems.

In one aspect, a tracking system is provided that learns classes of natural landmarks on articulated tools off-line. The system learns the landmarks by training an efficient multi-class classifier on a discriminative feature descriptor from manually ground-truthed data. The classifier is run on a new image frame to detect all extrema representing the location of each feature type, where confidence values and geometric constraints help to reject false positives. Next, stereo matching is performed with respect to the corresponding camera to recover 3D point locations on the tool. By knowing a priori the locations of these landmarks on the tool part (from the tool's CAD model), the pose of the tool is recovered by applying a fusion algorithm of kinematics and these 3D locations over time and computing the most stable solution of the configuration. Multiple tools are handled simultaneously by applying a tool association algorithm and the system of the presently disclosed subject matter is able to detect features on different types of tools. The features detected are small-scaled (˜2% of the image), vary in the amount of texture, and are observed under many different perspective views. The features are designed to be used within a marker-less pose estimation framework which fuses kinematics with vision, although this is out-of-the-scope of the current paper. The learning system of the presently disclosed subject matter is extends to multiple tool types and multiple tools tracked simultaneously as well as various types of surgical data.

The da Vinci® surgical robot is a tele-operated, master-slave robotic system. The main surgical console is separated from the patient, whereby the surgeon sits in a stereo viewing console and controls the robotic tools with two Master Tool Manipulators (MTM) while viewing stereoscopic high-definition video. The patient-side hardware contains three robotic manipulator arms along with an endoscopic robotic arm for the stereo laparoscope. A typical robotic arm has 7 total degrees-of-freedom (DOFs), and articulates at the wrist. The stereo camera system is calibrated for both intrinsics and stereo extrinsics using standard camera calibration techniques. Although the cameras have the ability to change focus during the procedure, a discrete number of fixed focus settings are possible, and camera calibration configurations for each setting are stored and available at all times, facilitating stereo vision approaches as described below.

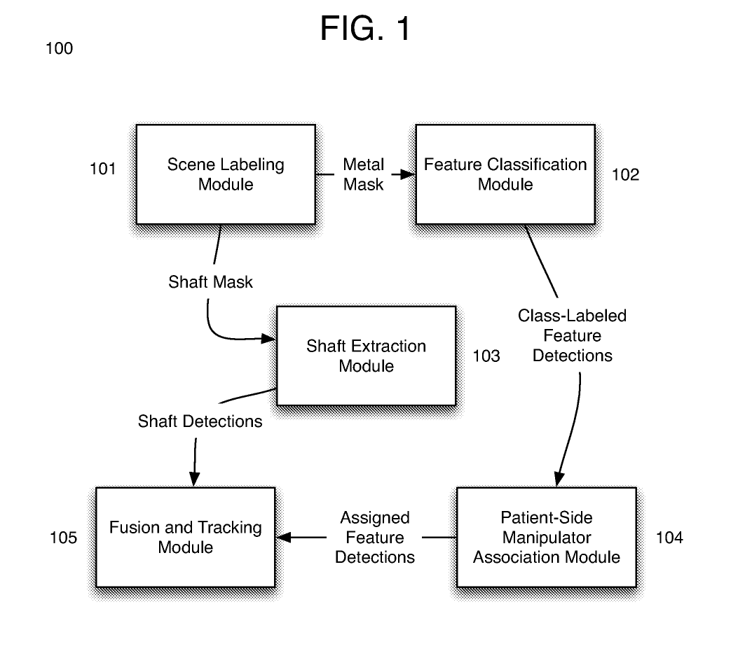

FIG. 1 provides an overview of the modules and algorithm of a detection and tracking system in accordance with an embodiment of the disclosed subject matter. Generally, the system includes a Scene Labeling Module 101 which applies a multi-feature training algorithm to label all pixels in an image of an anatomical scene with medical tool(s), a Feature Classification Module 102 which uses a classifier on feature descriptors to localize known landmarks on the tool tips, and a Shaft Extraction Module 103 that uses a shaft mask from the Scene Labeling Module 101 to fit cylinders to the shaft pixels in the image for all visible tools, whenever possible. A Patient-Side Manipulator (PSM) Association Module 104 uses class-labeled feature detections output from the Feature Classification Module 102 to determine which feature is associated with which tool in the image and a Fusion and Tracking Module 105 takes outputs from both the Shaft Extraction Module 103 and the Patient-Side Manipulator Association Module 104 to fuse visual observations with raw kinematics and track the articulated tools over time. In the paragraphs that follow, each of these modules is explained further.

### Scene Labeling Module

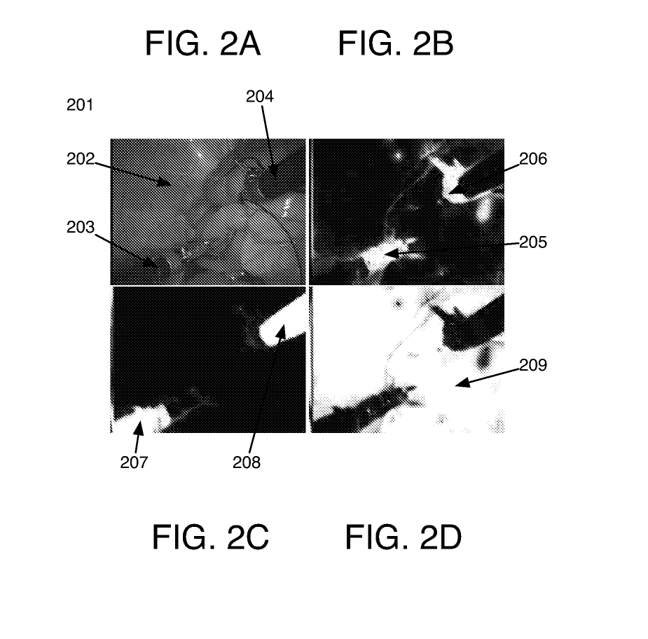

Scene Labeling Module 101 labels every pixel in an input image. Referring to FIG. 2A, the input image is the scene image 201, which typically includes the anatomical scene 202 and medical tool(s) 203 and 204. The scene is labeled with one of three classes: Metal, Shaft, or Background. A Gaussian Mixture Model (GMM) of several color and texture features is learned off-line for each of these three classes. Subsequently, a class-conditional probability is assigned for each of the classes to every pixel and a label is assigned.

FIG. 2 shows an example result of the pixel labeling routine described with reference to FIG. 1. FIG. 2A shows the original image 201 from an in-vivo porcine sequence of first and second robotic tools 203 and 204 performing a suturing procedure using the da Vinci® Surgical System. FIG. 2B shows the metal likelihood (e.g., tool tip, clevis), with mask regions 205 and 206 corresponding to the highest probability locations of metal. FIG. 2C shows the shaft likelihood, with mask regions 207 and 208 corresponding to the highest probability locations of shaft. FIG. 2D shows the background likelihood, with mask region 209 corresponding to the highest probability location of the background. The metal class represents all pixels located at the distal tip of the tool, from the clevis to the grippers. All of the features to be detected by the Feature Classification Module 102 are located in this region. Additionally, it is described below how the shaft class is used to fit a cylinder to the tool's shaft, whenever possible.

Typically, surgeries performed with the da Vinci® are quite zoomed in, and so the shaft is not usually visible enough to fit a cylinder (the typical approach to many tool tracking algorithms)). However, at times the camera is zoomed out and so this scene pixel labeling routine allows the algorithm to estimate the 6-DOF pose of the shaft as additional information. By estimating the approximate distance of the tool from the camera using stereo matching of sparse corner features on the tool's tip, it can be determined if the shaft is visible enough to attempt to fit a cylinder. When the camera is zoomed out, although the shaft is visible the features on the tool tip are not so easily detected. Therefore, recognition can be based on a combination of shaft features, tool-tip features, and a hybrid in between depending on the distance of the tool to the camera. These pixel labelings help to assist in both feature detection and shaft detection, as described further in the following text.

### Feature Classification Module

Feature Classification Module 102 analyzes only the pixels which were labeled as Metal by Scene Labeling Module 101 (mask regions 205 and 206 of FIG. 2B). This reduces both the false positive rate as well as the computation time, helping to avoid analyzing pixels which are not likely to be one of the features of interest (because they are known beforehand to be located on the tool tip). A multi-class classifier is trained using a discriminative feature descriptor. Class-labeled features are then localized in the image. Next, these candidate feature detections are stereo matched and triangulated to localize as 3D coordinates. These feature detection candidates are analyzed further using known geometric constraints to remove outliers and then are fed into the fusion and tracking stage of the algorithm.

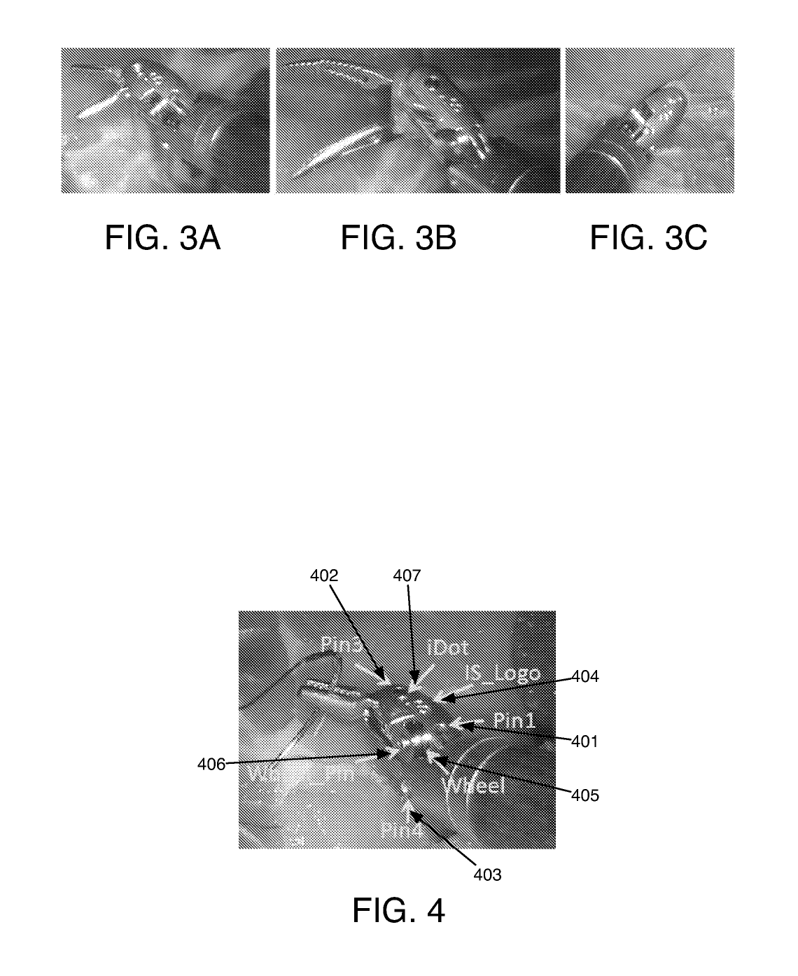

According to one aspect of the present subject matter, data is collected for the purposes of training the classifier. In one embodiment, nine different video sequences are used that span various in-vivo experiments, to best cover a range of appearance and lighting scenarios. For training, a Large Needle Driver (LND) tool can be used (shown in FIG. 3A). However, as discussed below, this will extend well to other types of tools, such as the Maryland Bipolar Forceps (MBF) (shown in FIG. 3B) and Round Tip Scissors (RTS) (shown in FIG. 3C). With training only on the Large Needle Driver, the system of the present disclosure is able to track on the Large Needle Driver, Maryland Bipolar Forceps and Round Tip Scissors. Seven naturally-occurring landmarks are manually selected as shown in FIG. 4 overlain on an image of the LND. The features chosen are of the pins that hold the distal clevis together 401, 402 and 403, the IS logo in the center 404, the wheel 405, wheel pin 406, and the iDot 407. From time-to-time this combination of landmarks is referred to as a marker pattern, Mi. The features chosen may also include known, invariant locations on the mid-line of the shaft axis to this marker pattern to be used in the fusion module.

For each frame in the ground truth procedure, the best encompassing bounding-box is manually dragged around each feature of interest, to avoid contamination from pixels which don't belong to the tool. To obtain as large a dataset as possible with reasonable effort, some embodiments of the presently disclosed subject matter coast through small temporal spaces using Lucas-Kanade optical flow (KLT) to predict ground truth locations between user clicks as follows: the user drags a bounding-box around a feature of interest; the software uses KLT optical flow to track this feature from frame-to-frame (keeping the same dimensions of the box); as the user inspects each frame, if either the track gets lost or the size changes, the user drags a new bounding-box and starts again until the video sequence ends.

This allows for faster ground truth data collection while still manually-inspecting for accurate data. Overall, a training set can comprise ˜20,000 total training samples across the seven feature classes.

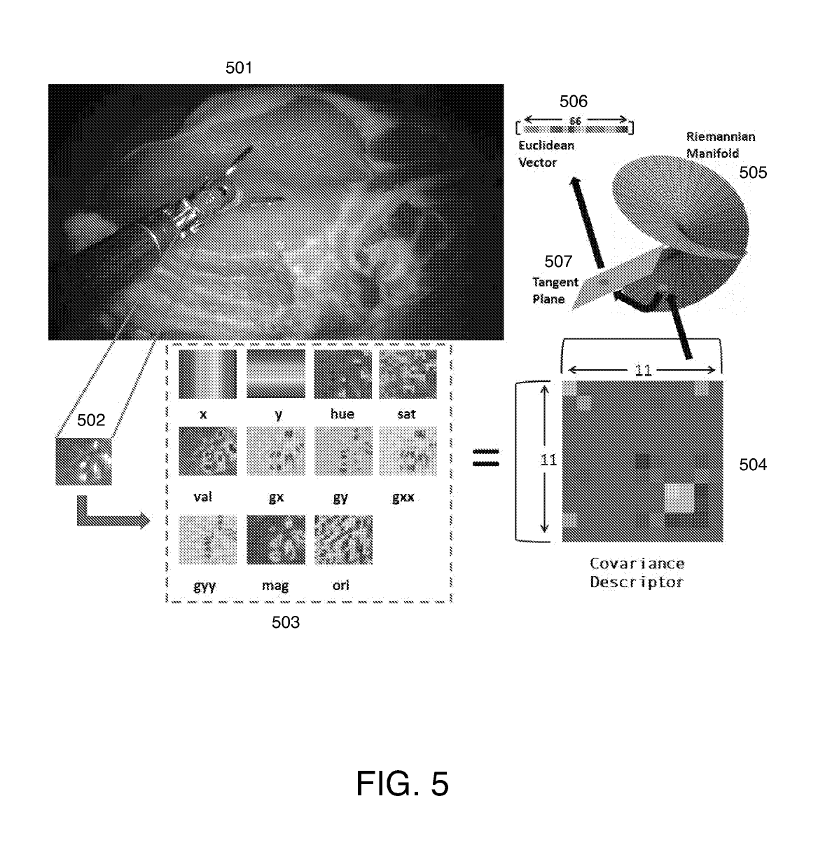

A feature descriptor capable of discriminating these feature landmarks from each other robustly is disclosed. A discriminative and robust region descriptor to describe the feature classes is required because each feature is fairly small (e.g., 17-25 pixels wide, or ˜2% of the image). In one embodiment of the present subject matter, a Region Covariance Descriptor is used, where the symmetric square covariance matrix of d features in a small image region serves as the feature descriptor (depicted in FIG. 5). Given an image I of size [W×H], d=11 features are extracted, resulting in a [W×H×d] feature image, as shown in equation (1), where x, y are the pixel locations; Hue, Sat, Val are the hue, saturation, and luminance values from the HSV color transformation at pixel location (x, y); Iₓ, I_{y}, are the first-order spatial derivatives: Iₓₓ, I_{yy} are the second-order spatial derivatives; and the latter two features are the gradient magnitude and orientation, respectively. The first two pixel location features are useful because their correlation with the other features are present in the off-diagonal entries in the covariance matrix. The [d×d] covariance matrix C_{R} of an arbitrary rectangular region R within F then becomes our feature descriptor.

$$F = [ xy Hue Sat Val {I}_{x} {I}_{y} {I}_{xx} {I}_{yy} \sqrt{{I}_{x}^{2} + {I}_{y}^{2}} arctan ( \frac{{I}_{y}}{{I}_{x}} ) ] \qquad ( 1 )$$

According to some embodiments of the present subject matter and as shown in FIG. 5, several independent features are combined compactly into a single feature descriptor. Eleven features are used overall (shown in the dotted box 503), specifically the (x, y) locations, hue/saturation/value color measurements, first and second order image gradients, and gradient magnitude and orientation. A rectangular region 502 (inset box shown zoomed from the original image 501) of the image 501 is described by using the covariance matrix of these 11 features within that region, yielding an 11×11 symmetric matrix 504. In order to use this matrix as a descriptor with typical linear mathematical operations, it must be mapped from it's natural Riemannian space 505 to a vector space using Lie Algebra techniques, yielding a 66-dimensional vector space descriptor 506, described in further detail below.

Each C_{R} can be computed efficiently using integral images. The sum of each feature dimension as well as the sum of the multiplication of every two feature dimensions is computed. Given these first and second order integral image tensors, the covariance matrix 504 of any rectangular region 502 can be extracted in O(d²) time. Using the ground truth data collected in accordance with the method given above, covariance descriptors of each training feature are extracted and the associated feature label is stored for training a classifier. However, the d-dimensional nonsingular covariance matrix descriptors 504 cannot be used as is to perform classification tasks directly because they do not lie on a vector space, but rather on a connected Riemannian manifold 505, and so the descriptors must be post-processed to map the [d×d] dimensional matrices C_{R} 540 to vectors cⱼε [symbol] ^{d(d+1)/2} 506.

Methods for post-processing the covariance descriptors to a vector space are known in the art. Symmetric positive definite matrices, of which the nonsingular covariance matrices above belong, can be formulated as a connected Riemannian manifold 505. A manifold is locally similar to a Euclidean space, and so every point on the manifold has a neighborhood in which a homeomorphism can be defined to map to a tangent vector space. According to one embodiment of the present subject matter, the [d×d] dimensional matrices above 504 are mapped to a tangent space 507 at some point on the manifold 505, which will transform the descriptors to a Euclidean multi-dimensional vector-space for use within the classifier according to the following method. Given a matrix X, the manifold-specific exponential mapping at the point Y is defined according to equation (2), and logarithmic mapping according to equation (3).

$$\begin{matrix}{exp}_{X} ( Y ) = {X}^{\frac{1}{2}} exp ( {X}^{- \frac{1}{2}} {YX}^{- \frac{1}{2}} ) {X}^{\frac{1}{2}} & ( 2 ) \\ {log}_{X} ( Y ) = {X}^{\frac{1}{2}} log ( {X}^{- \frac{1}{2}} {YX}^{- \frac{1}{2}} ) {X}^{\frac{1}{2}} & ( 3 )\end{matrix}$$

In these formulations, exp and log are the ordinary matrix exponential and logarithmic operations. An orthogonal coordinate system is defined at a tangent space with the vector operation. To obtain the vector-space coordinates at X for manifold point Y, the operation of equation (4) is perfumed, where upper extracts the vector form of the upper triangular part of the matrix. In the end, the result is a vector space with dimensionality

$$q = \frac{d ( d + 1 )}{2} .$$

$${vec}_{X} ( Y ) = upper ( {X}^{- \frac{1}{2}} {YX}^{- \frac{1}{2}} ) \qquad ( 4 )$$

The manifold point at which a Euclidean tangent space is constructed is the mean covariance matrix of the training data. To compute the mean matrix μ_{C}_{_{R}} in the Riemannian space, the sum of squared distances is minimized according to equation (5). This can be computed using the update rule of equation (6) in a gradient descent procedure. The logarithmic mapping of Y at μ_{C}_{_{R}} is used to obtain the final vectors. The training covariance matrix descriptors are mapped to this Euclidean space and are used to train the multi-class classifier, described below.

$$\begin{matrix}{μ}_{{C}_{R}} = {argmin}_{Y ∈ M} {\sum }_{i = 1}^{N} {d}^{2} ( {X}_{i} , Y ) & ( 5 ) \\ {μ}_{{C}_{R}}^{t + 1} = {exp}_{{μ}_{{C}_{R}}^{t}} [ \frac{1}{N} {\sum }_{i = 1}^{N} {log}_{{μ}^{t}} ( {X}_{i} ) ] & ( 6 )\end{matrix}$$

Various multi-class classifiers known in the art may suit this problem. However, runtime is an important factor in the choice of a learning algorithm to be used in accordance with the present subject matter. Consequently, in one embodiment of the present disclosure, multi-class classification is performed using a modified Randomized Tree (RT) approach. In addition to providing feature labels, the approach of the present disclosure, allows retrieval of confidence values for the classification task which will be used to construct class-conditional likelihood images for each class. Various feature descriptors, such as Scale-invariant Feature Transforms (SIFT), Histograms-of-Oriented Gradients (HoG), and the Covariance Descriptors previously discussed may be paired with various classification algorithms such as Support Vector Machines (SVM) or the two variants on RTs, described below. This results in a total of nine possible descriptor/classifier combinations: SIFT/SVM, SIFT/RT, SIFT/BWRT, HoG/SVM, HoG/RT, HoG/BWRT, Covar/SVM, Covar/RT, and Covar/BWRT. In one embodiment of the present disclosure, the Covariance Descriptor is paired with the adapted RTs to achieve a sufficient level of accuracy and speed.

SIFT has been used as a descriptor for feature point recognition/matching and is often used as a benchmark against which other feature descriptors are compared. It has been shown that SIFT can be well approximated using integral images for more efficient extraction. In one embodiment of the present disclosure, ideas based on this method may be used for classifying densely at many pixels in an image.

HoG descriptors describe shape or texture by a histogram of edge orientations quantized into discrete bins (in one embodiment of the present disclosure, 45 are used) and weighted on gradient magnitude, so as to allow higher-contrast locations more contribution than lower-contrast pixels. These can also be efficiently extracted using integral histograms.

An SVM constructs a set of hyperplanes which seek to maximize the distance to the nearest training point of any class. The vectors which define the hyperplanes can be chosen as linear combinations of the feature vectors, called Support Vectors, which has the effect that more training data may produce a better overall result, but at the cost of higher computations. In an alternative embodiment of the present disclosure using SVM, Radial Basis Functions are used as the kernel during learning.

RTs naturally handle multi-class problems very efficiently while retaining an easy training procedure. The RT classifier Λ is made up of a series of L randomly-generated trees Λ=[γ₁, . . . , γ_{L}], each of depth m. Each tree γᵢ, for iε1, . . . , L, is a fully-balanced binary tree made up of internal nodes, each of which contains a simple, randomly-generated test that splits the space of data to be classified, and leaf nodes which contain estimates of the posterior distributions of the feature classes.

To train the tree, the training features are dropped down the tree, performing binary tests at each internal node until a leaf node is reached. Each leaf node contains a histogram of length equal to the number of feature classes b, which in one embodiment of the present disclosure is seven (for each of the manually chosen landmarks shown in FIG. 4). The histogram at each leaf counts the number of times a feature with each class label reaches that node. At the end of the training session, the histogram counts are turned into probabilities by normalizing the counts at a particular node by the total number of hits at that node. A feature is then classified by dropping it down the trained tree, again until a leaf node is reached. At this point, the feature is assigned the probabilities of belonging to a feature class depending on the posterior distribution stored at the leaf from training.

Because it is computationally infeasible to perform all possible tests of the feature, L and m are chosen so as to cover the search space sufficiently and to best avoid random behavior. In one embodiment, L=60 trees are used, each of depth m=11. Although this approach is suitable for matching image patches, traditionally the internal node tests are performed on a small patch of the luminance image by randomly selecting 2 pixel locations and performing a binary operation (less than, greater than) to determine which path to take to a child. In one embodiment, feature descriptor vectors are used rather than image patches, and so the node tests are adapted to suit this specialized problem.

In one embodiment of the disclosed subject matter, for each internal tree node a random linear classifier hᵢ to feature vector x is constructed to split the data as shown in equation (7), where n is a randomly generated vector of the same length as feature x with random values in the range [−1, 1] and zε[−1, 1] is also randomly generated. This test allows for robust splitting of the data and is efficiently utilized as it is only a dot product, an addition, and a binary comparison per tree node. In this way, the tree is trained with vectorized versions of the covariance descriptors and build up probability distributions at the leaf nodes. The resulting RT classifier Λ is the final multi-class classifier. The results from each tree γᵢ are averaged across all L trees. However, even with relatively small values for L and m for computation purposes, the search space is still quite large given the appreciable amount of choices for randomly-created linear dot products at the internal tree nodes, and this leaves the training approach susceptible to randomness. To alleviate this, the approach is further modified from a conventional RT.

$${h}_{i} = \{ \begin{matrix}{n}^{T} x + z \leq & go to right child \\ otherwise & go to left child\end{matrix} \qquad ( 7 )$$

In one aspect of the present subject matter, an improved RT approach is disclosed, which is referred to as Best Weighted Randomized Trees (BWRT). Each tree γᵢ is essentially a weak classifier, but some may work better than others, and can be weighted according to how well they behave on the training data. Because of the inherent randomness of the algorithm and the large search space to be considered, an improvement is shown by initially creating a randomized tree bag Ω of size E>>L. This allows us initial consideration of a larger space of trees, but after evaluation of each tree in Ω on the training data, the best L trees are selected for inclusion in the final classifier according to an error metric.

The latter point allows consideration of more of the parameter space when constructing the trees while retaining the computational efficiency of RTs by only selecting the best performers. In order to evaluate a particular tree on the training data, the posterior probability distributions at the leaf nodes is considered. First, the training data is split into training and validation sets (e.g., ˜70% is used to train and the rest to validate). Next, all trees from the training set in Ω are trained as usual. Given a candidate trained tree \tilde{γ}ᵢεΩ, each training sample is dropped from the validation set through γᵢ until a leaf node is reached. Given training feature Xⱼ and feature classes 1, . . . , b, the posterior distribution at the leaf node contains b conditional probabilities p_{\tilde{γ}}_{ᵢ}(y|Xⱼ) where yε1, . . . , b. To evaluate the goodness of tree \tilde{γ}ᵢ on Xⱼ, p_{\tilde{γ}}_{ᵢ} (yⱼ|Xⱼ) is compared to the desired probability 1 of label yⱼ, and accumulate the root-mean squared (RMS) error of all training features Xⱼ across all validation trees in Ω. The top L trees (according to the lowest RMS errors) are selected for the final classifier Λ. In some embodiments, the initial bag size is E=125,000 candidate tree classifiers, cut down to L=60 trained trees for the final classifier.

In one aspect of the disclosed subject matter, in addition to selecting the best trees in the bag, the error terms are used as weights on the trees. Rather than allowing each tree to contribute equally to the final averaged result, each tree is weighted as one-over-RMS so that trees that label the validation training data better have a larger say in the final result than those which label the validation data worse. As such, for each γᵢΣΛ an associated weight wᵢ is computed such that wᵢ=1/rmsᵢ where rmsᵢ is the accumulated RMS error of tree γᵢ on the validation data. At the end, all weights wᵢ for iε1 . . . , L are normalized to sum to 1 and the final classifier result is a weighted average using these weights.

Given the trained classifier Λ, features for each class label are detected on a test image by computing dense covariance descriptors C_{R} (e.g., at many locations in the image) using the integral image approach for efficient extraction. Each C_{R} is mapped to a vector space using the mean covariance μ_{C}_{_{R}} of the training data as previously described, producing a Euclidean feature cⱼ. Each cⱼ is dropped through the trees γᵢ and the probabilities are averaged at the obtained leaf nodes to get a final probability distribution p_{L}, representing the probability of cⱼ belonging to each of the L feature classes. This results in L class-probability images. The pixel locations are obtained by non-maximal suppression in each class-probability image.

The probabilities are used instead of the classification labels because a classification of label l arises when its confidence is greater than all other b−1 classes in the classifier. However, a confidence of 95% for one pixel location means more than a confidence of 51% for that same labeling at a different location. In this case, the pixel with the higher probability would be chosen (even given they both have the same label), and for this reason detect is performed in probability space rather than in labeling space.

After candidate pixel locations are determined for each feature class, the feature detections are stereo matched in the corresponding stereo camera using normalized cross-correlation checks along the epipolar line, the features are triangulated to retrieve 3D locations. Using integral images of summations and squared-summations correlation windows along these epipoles are efficiently computed.

### Patient-Side Manipulator (PSM) Association Module

Referring back to FIG. 1, after deriving the 3D point locations (in the camera's coordinate system) and associated feature labels, Patient-Side Manipulator (PSM) Association Module 104, determines with which tool each feature is associated. With multiple tools in the scene, it is unclear after determination of class-labeled 3D feature locations which feature is associated with which tool. Typically, the da Vinci® has three Patient-Side Manipulators (PSMs), only two of which are visible in the camera frame at any time. The manipulators are referred to as PSM₀, PSM₁, and PSM₂. For example and not limitation, a case in which two tools (PSM₀ and PSM₁) simultaneously appear is discussed below. In this case, Patient-Side Manipulator (PSM) Association Module 104 associates feature detections with PSMs.

Each PSM has a marker pattern, M₀ and M₁, respectively, each in their zero-coordinate frame (e.g., the coordinate system before any kinematics are applied to the marker). Using the forward kinematics estimate from each PSM, the marker patterns are rotated to achieve the estimated orientations of each PSM. The full rigid-body transform from the forward kinematics is not applied because most of the error is in the position, and although the rotation isn't fully correct, it's typically close enough to provide the geometric constraints require. This leaves equations (9) and (10), where Rot₀ and Rot₁ are the 3×3 rotation matrices from the full rigid-body transformations representing the forward kinematics for PSM₀ and PSM₁, respectively. Given \tilde{M}₀ and \tilde{M}₁, 3D unit vectors are computed between each of the rotated point locations within each marker. This yields 7×7 3D unit vectors in a 7×7×3 matrix for each rotated marker pattern. Additionally, a 7×7 distance matrix Dm is calculated between each marker location in its zero-coordinate frame.

\tilde{M}₀=Rot₀(M₀) (9)

\tilde{M}₁=Rot₁(M₁) (10)

Next, given N detected feature observations using the classification method described above, both an N×N distance matrix between each 3D feature observation and an N×N×3 matrix of unit vectors are computed, similar to those computed for the marker patterns using the kinematics estimates from the robot. Finally, any feature observations which do not adhere to one of the pre-processed marker distance and rotation configurations according to the PSMs are rejected. Using empirically determined distance (e.g., ˜3-5 mm) and orientation (e.g., ˜10°-20°) thresholds, the PSM associated with each feature is determined, allowing only one assignment per feature class to each PSM.

### Shaft Extraction Module

Referring back to FIG. 1, Shaft Extraction Module 103 determines the location of the shaft in an input image. As noted above, it is not guaranteed that there are enough shaft pixels visible to compute valid cylinder estimates, and so in one embodiment of the present disclosure, stereo vision is used to estimate the distance of the tool tip to the camera. If the algorithm determines that the tools are situated far enough away from the camera so that the shaft is sufficiently visible, the shaft likelihood mask as provided by the Scene Labeling Module 101 is used to collect pixels in the image (potentially) belonging to one of the two tools' shafts. Assuming that each tool shaft is represented as a large, rectangular blob, using connected components and 2D statistical measures (e.g., aspect ratios, total pixel areas) those areas of the image which are not likely to be one of the tool shafts are eliminated.

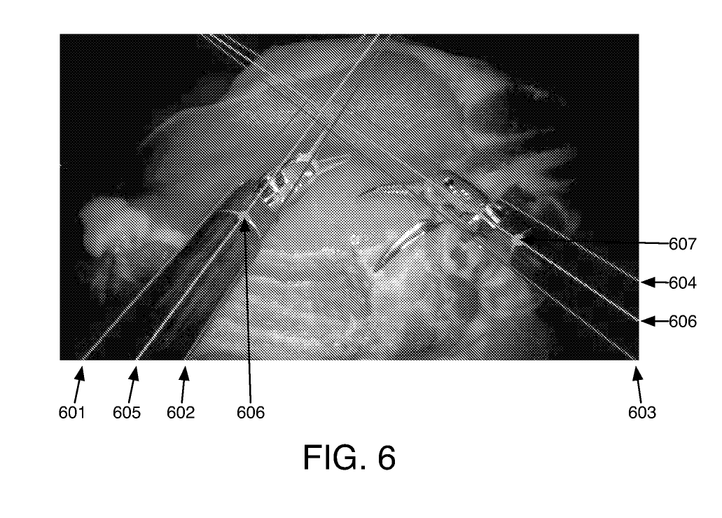

As shown in FIG. 6, 2D boundary lines 601, 602, 603, and 604 are fitted to each candidate shaft blob. By extracting the boundary lines of the shaft (outer pairs of lines 601-602 and 603-604), the mid-line axis (inner lines 605 and 606), and then the intersection location between the tool's shaft and the clevis (dots 607 and 608 on inner lines 605 and 606), shaft observations are provided to the Fusion and Tracking Module 105 along with the feature observations. Using projective geometry a 3D cylinder is fit to each pair of 2D lines, representing a single tool's shaft. Then, the intersection point in the 2D image where the tool shaft meets the proximal clevis is located by moving along the cylinder axis mid-line from the edge of the image and locating the largest jump in gray-scale luminance values, representing where the black shaft meets the metal clevis (dots 607 and 608 on inner lines 605 and 606). A 3D ray is projected through this 2D shaft/clevis pixel to intersect with the 3D cylinder and localize on the surface of the tool's shaft. Finally, this 3D surface location is projected onto the axis mid-line of the shaft, representing a rotationally-invariant 3D feature on the shaft. This shaft feature is associated with its known marker location and is added to the fusion stage 105 along with the feature classification detections.

### Fusion and Tracking Module

Because features detected are not guaranteed to always be visible in any given frame, the robot kinematics are combined with the vision estimates in Fusion and Tracking Module 105 to provide the final articulated pose across time. The kinematics joint angles are typically available at a very high update rate, although they may not be very accurate due to the error accumulation at each joint.

For surgical robots like the da Vinci®, it is important to keep the instrument insertion point (also termed remote center) stationary. This means that one part of the robotic arm holding the instrument does not move during the surgery (e.g., it is passive). The error of the end effector pose comes from both the error in zero calibration of the potentiometers at the joints and the error in the kinematics chain due to the link lengths. These are mostly static because the errors from the passive setup joints have more influence on the overall error as they are further up in the kinematic chain and have longer link lengths than the active joints. Therefore, by solving for this constant error bias, it can be applied to the raw kinematics of the active joints to determine fairly accurate overall joint angle estimates. This bias essentially amounts to a rigid body pose adjustment at the stationary remote center. Although there is also error for the robotic arm holding the camera, when it does not move it is not necessary to include this in the error contributions.

To perform these adjustments on-line, an Extended Kalman Filter (EKF) is used. The state variables for the EKF contain entries for the offset of the remote center, which is assumed to be either fixed or slowly changing and so can be modeled as a constant process. The observation model comes from our 3D point locations of our feature classes. At least 3 non-collinear points are required for the system to be fully observable. The measurement vector is given in equation (11). The observation function which transforms state variables to observations is not linear, and so the Jacobians of equations (12) and (13) are required, where p^{K} is a 3D point location in the kinematics remote center coordinate frame KCS, qᵢ^{K} is a unit quaternion rotation between the true instrument joint coordinate system ICS and the KCS, and cᵢ^{K} is the remote center location in the KCS.

$$\begin{matrix}{y}_{3} = {[ {x}_{1} , {y}_{1} , {z}_{1} , … , {x}_{n} , {y}_{n} , {z}_{n} ]}^{T} & ( 11 ) \\ {J}_{1} = \frac{\partial {p}^{K}}{\partial {q}_{1}^{K}} & ( 12 ) \\ {J}_{2} = \frac{\partial {p}^{K}}{\partial {c}_{1}^{K}} & ( 13 )\end{matrix}$$

It is unlikely that any realistic solution to a computer vision problem does not contain outliers. The image analysis is of principal concern as it is input to the fusion and tracking module 105. To address this issue, in some embodiments of the present disclosure, an initial RANSAC phase is added to gather a sufficient number of observations and perform a parametric fitting of the rigid transformation for the pose offset of the remote center. This is used to initialize the EKF and updates online as more temporal information is accumulated. In some embodiments, a minimum of ˜30 total inliers are required for a sufficient solution to begin the filtering procedure. The rigid body transformation offset is computed using the 3D correspondences between the class-labeled feature observations, done separately for each PSM after the PSM association stage described above, and the corresponding marker patterns after applying the forward kinematics estimates to the zero-coordinate frame locations for each tool. Because the remote center should not change over time, this pose offset will remain constant across the frames, and so by accumulating these point correspondences temporally, a stable solution is achieved.

In some embodiments of the present disclosure, not all of the modules of FIG. 1 are present. For example, in one embodiment, Scene Labeling Module 101 and Shaft Extraction Module 103 are omitted and the input image is provided as input directly to the Feature Classification Module 102. In another embodiment, kinematics data is not used and so the Fusion and Tracking Module 105 is omitted and the pose of the Patient Side Manipulator is determined based on the output of the feature classification module. In another embodiment, there is only one Patient Side Manipulator, and the Patient Side Manipulator Association Module 104 is omitted. Other combinations of the modules of FIG. 1 that do not depart from the spirit or scope of the disclosed subject matter will be apparent to those of skill in the art.

### EXPERIMENTAL RESULTS

The system of the present disclosure has been demonstrated to work on two types of datasets, both collected previously on a da Vinci® surgical robot: (1) porcine data (in-vivo), and (2) pork data (ex-vivo). The data which was used to test was specifically not included in the training collection procedure described above. After collecting and training the seven feature classes using ˜20,000 training samples with the Best Weighted Randomized Trees approach described above, the PSM association and geometric constraints method described above was applied, and finally the fusion and tracking stage was performed.

Overall, testing included 6 different video sequences, totaling 6876 frames (458 seconds worth of video). Each video frame had two tools visible at all times. Across these video sequences, three different types of da Vinci® tools, were analysed, the Large Needle Driver (shown in FIG. 3A), Maryland Bipolar Firceps (shown in FIG. 3B) and Round Tip Scissors (shown in FIG. 3C). The system was trained only on the Large Needle Driver (LND) (shown in FIG. 3A), and tested on that same LND tool in addition to the Maryland Bipolar Forceps (MBF) (shown in FIG. 3B) and Round Tip Scissors (RTS) (shown in FIG. 3C). The method works on these other tools because there are many shared parts across the tools, including the pins used to hold the clevis together and the IS logo in the center of the clevis. Even though the overall appearance of each tool is quite different, results show that the method extends very well to different tools given that the lower-level features are consistent. However, if newer tools are introduced which don't share these parts in common, more training data and feature classes must be considered and included in training the classifier discussed above.

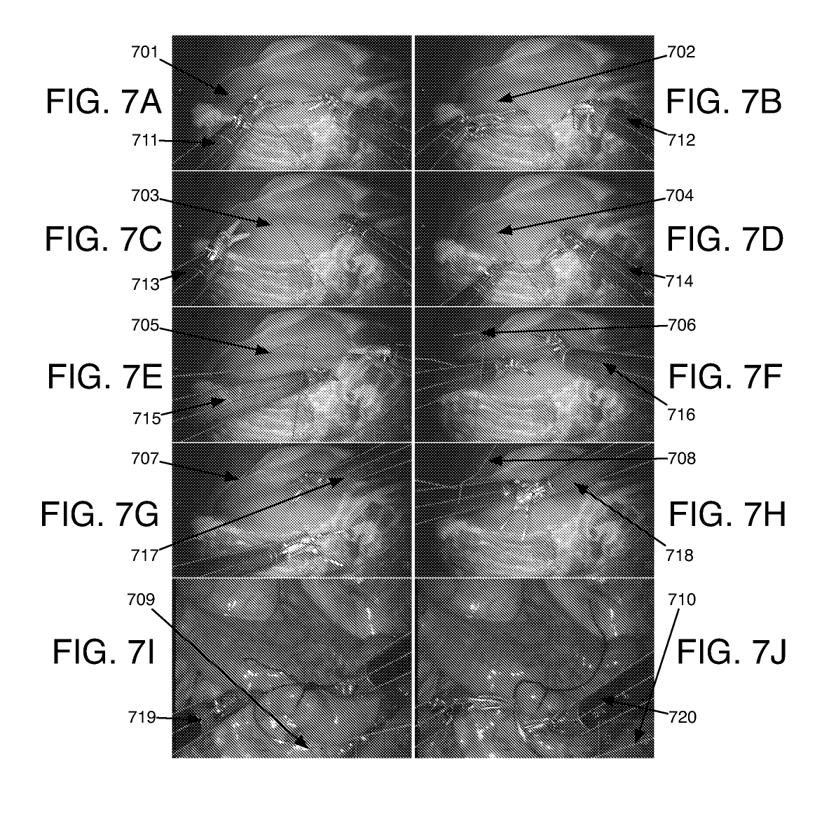

Ten sample results are shown in FIGS. 7A-J from various test sequences. FIGS. 7A-H show ex-vivo pork results with different combinations of the LND, MBF, and RTS tools. FIGS. 7I-J show a porcine in-vivo sequence with an MBF on the left and an LND on the right. In FIG. 7H, one tool is completely occluding the other tool's tip, however the EKF from the Fusion stage assists in predicting the correct configuration. For each, superposed lines 701-710 portray the raw kinematics estimates as given by the robot, projected into the image frames. The lines 711-720 superposed on the tools show the fixed kinematics after running application of the detection and tracking system of the present disclosure. FIGS. 7A-B show the MBF (left) and LND (right). FIGS. 7C-D show the RTS (left) and MBF (right). FIGS. 7E-F show the LND (left) and RTS (right). FIGS. 7G-H show the MBF (left) and MBF (right). FIGS. 7I-J show the MBF (left) and LND (right). The significant errors are apparent, where in some images the estimates are not visible at all, motivating the need for the system and methods of the present disclosure. A visual inspection yields a fairly accurate correction of the kinematics overlaid on the tools.

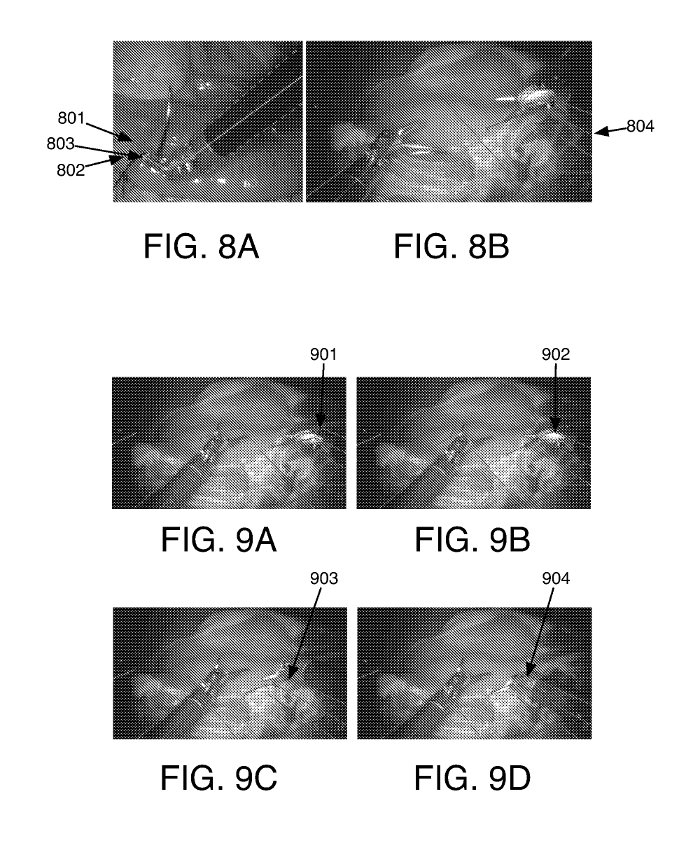

Because joint-level ground truth for articulated tools is very difficult to collect accurately and on a large dataset, the accuracy of the tracking system of the present disclosure is evaluated in the 2D image space. FIG. 8A depicts the evaluation scheme for the kinematics estimates. The dotted lines 801, 802 define an acceptable boundary for the camera-projection of the kinematics, where the solid line 803 is a perfect result. FIG. 8B shows an example of an incorrect track 804 on the right-most tool. Using this scheme, each frame of the test sequences was manually inspected, and resulted in a 97.81% accuracy rate over the entire dataset.

TABLE 1 shows a more detailed breakdown of the evaluation. Overall, the system of the present disclosure was tested against 6 sequences, including both ex-vivo and in-vivo environments, all with two tools in the scene. TABLE 1 shows the test sequence name in the first (leftmost) column, the number of tracks labeled as correct in the second column, the total possible number of detections in that sequence in the third column, and the final percent correct in the last (rightmost) column. Note that in any given frame, there may be 1 or 2 tools visible, and this is how the numbers in the third column for the total potential number of tracks in that sequence are computed. Finally, the last row shows the total number of correct tracks detected as 13315 out of a total possible of 13613, yielding the final accuracy of 97.81% correct. Also note that the accuracy was very similar across the sequences, showing the consistency of the system and methods of the present disclosure. Although the accuracy was evaluated in the 2D image space, this may not completely represent the overall 3D accuracy as errors in depth may not be reflected in the perspective image projections.

TABLE 1

| Sequence | # Correct | Potential | % Correct |
|---|---|---|---|
| Seq. 1 | 1890 | 1946 | 97.12% |
| Seq. 2 | 2114 | 2182 | 96.88% |
| Seq. 3 | 1447 | 1476 | 98.04% |
| Seq. 4 | 1611 | 1648 | 97.75% |
| Seq. 5 | 4376 | 4431 | 98.76% |
| Seq. 6 | 1877 | 1930 | 97.25% |
| TOTAL | 13315 | 13613 | 97.81% |

The full tracking system of the present disclosure runs at approximately 1.0-1.5 secs/frame using full-sized stereo images (960×540 pixels). The stereo matching, PSM association, and fusion/EKF updates are negligible compared to the feature classification and detection, which takes up most of the processing time. This is dependent on the following factors: number of trees in A, depth of each tree γᵢ, number of features used in the Region Covariance descriptor C_{R} (in one embodiment of the present disclosure, 11 are used, but less could be used), and the quality of the initial segmentation providing the mask prior. However, by half-sizing the images a faster frame-rate can be achieved (0.6-0.8 sees/frame, an example of which is shown in Seq. 5) while achieving similar accuracy. Also, because a solution is found for a remote center bias offset which remains constant over time, the frames can be processed at a slower-rate without affecting the overall accuracy of the tracking system. Finally, many stages of the classification are parallelizable, and both the Covariance Descriptor and Randomized Trees can be implemented on a GPU processor. Test results on the covariance processing show a reduction in the processing time of the feature tensors (equation (1)) from ˜700 ms to ˜100 ms, and this can be reduced further.

There many variations are possible in the implementation of a tracking system in accordance with the present disclosure. One such variation is found in the size of the window to use when extracting covariance descriptors for classification throughout the image. The reason is that, during training, the best encompassing bounding box around each feature is used, and the descriptors are well tuned to representing the entire feature. When applying the classifier, if the window is too small or too large, the descriptors won't capture the features well. To alleviate this, prior knowledge of the 3D sizes of the features is used to guide computation of the optimal window size. Using the stereo vision approach which determines if the shaft is visible enough to extract (as discussed above) and estimating that the features are ˜3×3 mm in size, the optimal window size in the image can be automatically determined dynamically on each frame. To further reduce errors, at every pixel location that is evaluated, a bounding box is extracted that is both full and half-sized according to this automatically determined window size to account for the smaller features (e.g., the pins). This improves the overall feature detection system.

Upon further inspection of the errors encountered during evaluation on the test sequences, it is found that most of the incorrect fixed/tracked kinematic configurations are due to a latency in the raw kinematics which causes the video and raw kinematics to be out-of-sync from time-to-time. This situation is shown more precisely in FIGS. 9A-D, which shows an example of kinematic latency in the right tool. Often the kinematics and video get out-of-sync with each other. Most of our errors are due to this fact, manifesting in the situation shown in FIGS. 9A-D. The four frames of FIGS. 9A-D are consecutive to each other in order. In FIG. 9A (representing time t), both tools are tracked well (as shown by lines 901). Then, in FIG. 9B (representing time t+1) and FIG. 9C (representing time t+2), the kinematics and video become out-of-sync and the right tool becomes inaccurately tracked (as shown by lines 902 and 903). However, in FIG. 9D (representing time t+3), the tools are tracked successfully again (as shown by lines 904). Looking at the overlay configuration 903 in FIG. 9C, which is essentially the same as the correct one immediately following in FIG. 9D (904), suggests this latency is the source of our errors. For the individual frames which had incorrect projections (according the scheme described above), the result would jump immediately to a correct configuration instead of getting lost completely, and the previous incorrect projection was in the location and configuration that the upcoming projection would eventually reach. Therefore, by logging the test data more precisely so that the video and kinematics are more in sync with each other, the accuracy would be expected to increase even further. However, in practice on a live system this kinematic latency does not exist.

The majority of tool tracking approaches in the literature work by estimating the cylinder of the shaft which is visible in the scene). However, as previously discussed, surgeons tend to work quite zoomed in, making this cylinder-fitting procedure very difficult, if not impossible, due to the limited number of visible shaft pixels. The remaining minority approaches work by analyzing the tip of the tool using features, however these will fail when the tool tip is too far away to be seen well by the camera. The approach described above is advantageous in that it dynamically decides which of these two approaches is optimal at any given time, and often uses both simultaneously to best track the tool over longer periods of time. Also, by using the pixel labeling method described above, the system of the present disclosure is able to tell more accurately when parts of the tool are occluded. For example, if the metal tool tip is occluded then the pixel labeling won't label the incorrect pixels from the occluder as metal, and false positives will be avoided. Occlusion errors will similarly be avoided for the shaft.

The present disclosure provides a tool detection and tracking framework which is capable of tracking multiple types of tools and multiple tools simultaneously. The algorithm has been demonstrated on the da Vinci® surgical robot, and can be used with other types of surgical robots. High accuracy and long tracking times across different kinds of environments (ex-vivo and in-vivo) are shown. By learning low-level features using a multi-class classifier, the system of the present disclosure overcomes different degrees of visibility for each feature. The hybrid approach of the present disclosure, using both the shaft and features on the tool tip, is advantageous over either of these methods alone. Using knowledge of the distance of the tool the system of the present disclosure can dynamically adapt to different levels of information into a common fusion framework. Finally, by fusing vision and kinematics, the system of the present disclosure can account for missed observations over time.

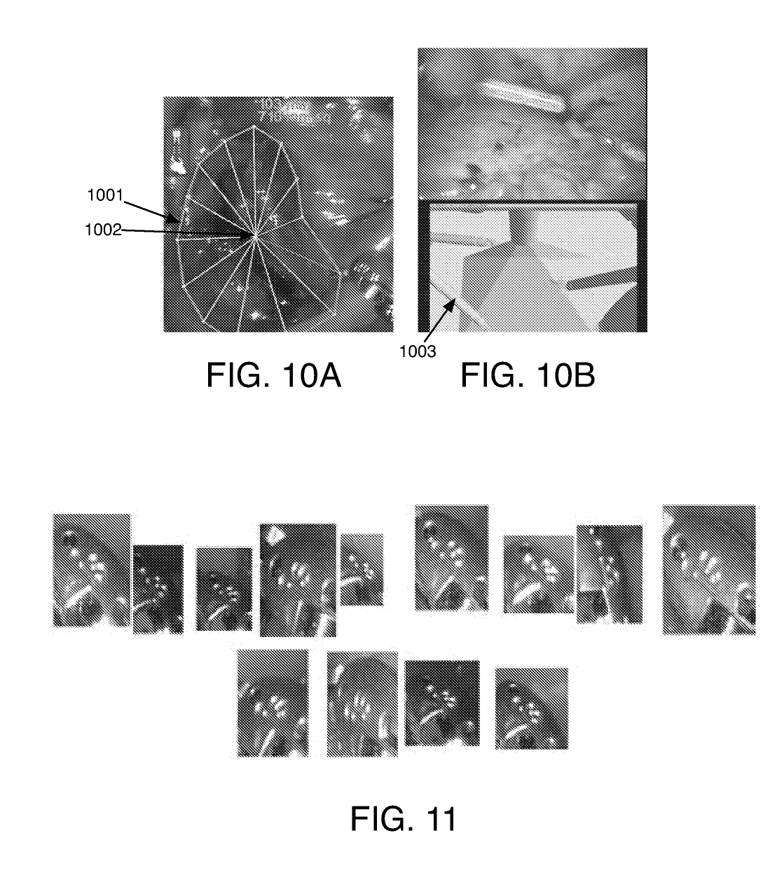

Example applications of tool tracking in accordance with the present disclosure are shown in FIGS. 10A-B. In FIG. 10A, a picture of a measurement tool measuring the circumference 1001 and area 1002 of a mitral valve is shown. In FIG. 10B, an example scenario of a lost tool (e.g., outside the camera's field-of-view) is shown, whereby the endoscopic image (top) shows only two tools, and with fixed kinematics and a graphical display (bottom), the surgeon can accurately be shown where the third tool 1003 (out of the left-bottom corner) is located and posed so they can safely manipulate the tool back into the field-of-view.

FIG. 11 depicts example appearance changes typically encountered of the IS Logo feature through different lighting and perspective effects, to motivate the need for a robust descriptor.

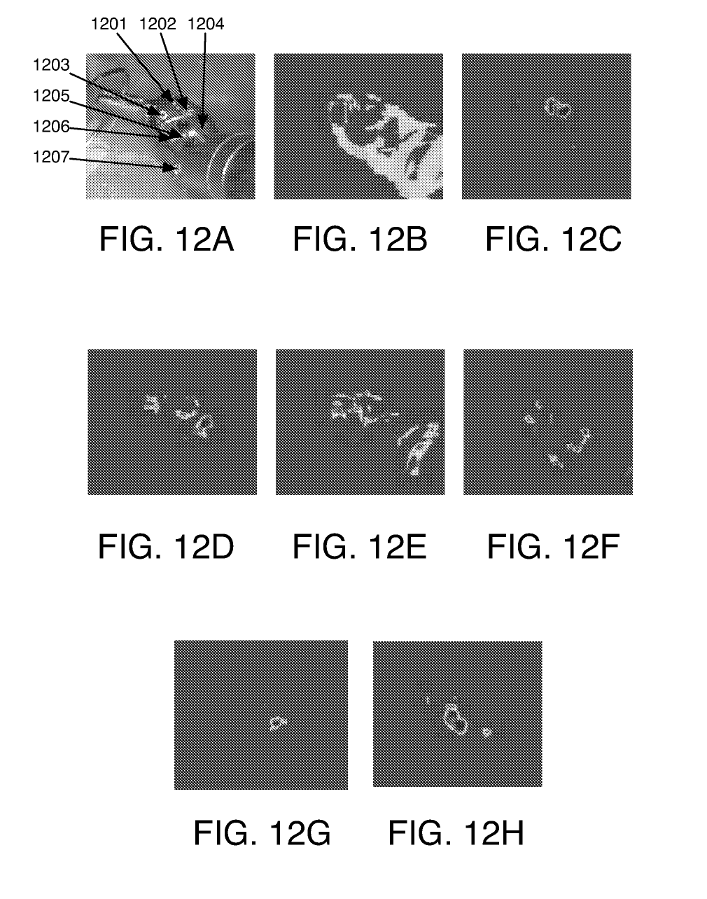

Although in one embodiment of the present disclosure, the Covariance Descriptor is paired with Best Weighted Randomized Trees to achieve a sufficient level of accuracy and speed, alternative combinations of descriptors and classifiers can be used. One method of evaluating available parings using the likelihood-space works as follows: given a test image, the multi-class classifier is run through the entire image, resulting in b probabilities at each pixel for each feature class. This yields b different likelihood images. In each likelihood, non-maximal suppression is performed to obtain the 3 best peaks in the likelihood. Then, a feature classification is marked correct if any of the 3 peaks in the likelihood is within a distance threshold (for example, 1% of the image size) of the ground truth for that feature type. This method is suitable because it is often the case that there is a local peak at the correct location for a feature, but it is not always the global peak. Therefore, in a full tracking system a temporal coherence filter can eliminate these outliers. FIGS. 12A-H show sample likelihoods on the tip of the LND tool overlain with extrema locations. FIG. 12A depicts the individual features with circles (from top to bottom, iDot 1201, IS Logo 1202, Pin3 1203, Pin1 1204, Wheel 1205, Wheel Pin 1206, Pin 4 1207). Six of the seven features are correctly detected as peaks in the class-conditional likelihoods (FIG. 12B—iDot, FIG. 12C—IS Logo, FIG. 12D—Pin1, FIG. 12F—Pin4, FIG. 12G—Wheel, FIG. 12H—Wheel Pin), where the Pin3 (FIG. 12E) feature is incorrectly detected. This was produced using the Covar/RT approach.

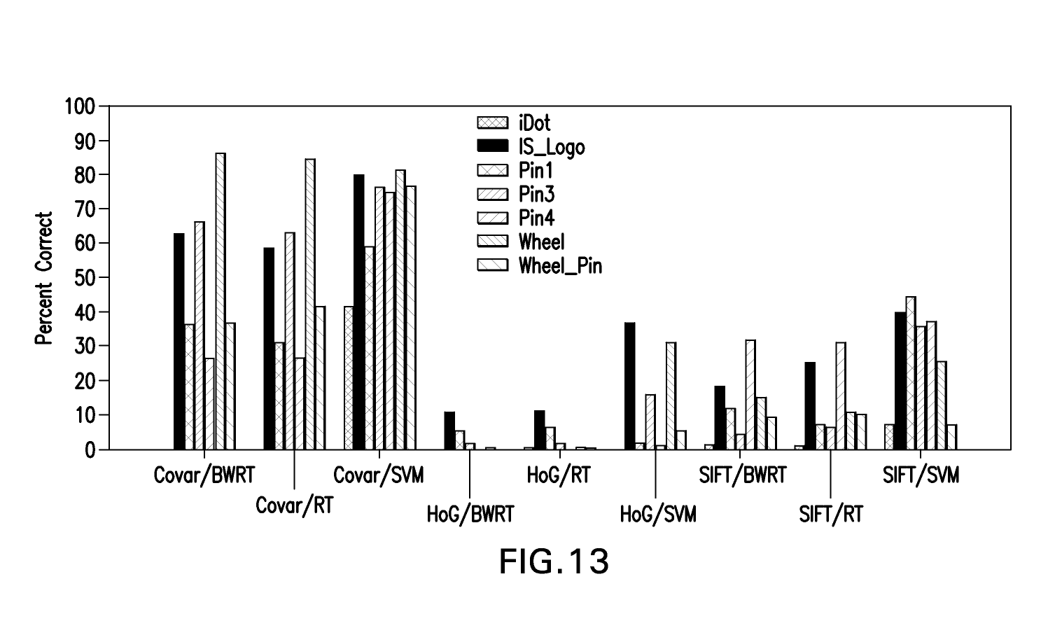

To evaluate accuracy, testing was performed on video which was specifically not used in the training stage. Testing was performed on 1500 frames from in-vivo sequences, which resulted in ˜4500 possible feature detections which were ground-truthed. The accuracy against the ground truth is shown in FIG. 13 for each individual feature type, separately. It is clear that different features are more reliably detected than others, which may be attributed to differences in size, texture, and uniqueness. However, it is obvious from this graph that the Region Covariance out-performs both the SIFT and HoG descriptors, regardless of the learning algorithm.

A more detailed analysis reveals that the SVM evaluates best overall, although both RT and BWRT are certainly comparable as different features perform differently. For example, Covar/SVM classifies the Wheel feature with 81% accuracy, whereas Covar/RT classifies that same feature at 84% and Covar/BWRT at 86%. Contrastly, Covar/SVM classifies the IS Logo feature at 80% against a classification rate of 59% for Covar/RT and 63% for Covar/BWRT.

The maximum achieved accuracy using the SIFT descriptor was 44% using SIFT/SVM on the Pin1 feature. Using the HoG descriptor, the best achieved accuracy was 37% using HoG/SVM on the IS Logo feature.

In addition to accuracy, the per-frame processing time of each algorithm is considered. As mentioned previously, SVMs tend to become more complex and time consuming as more support vectors are added, which arises due to more training data. Conversely, the tree approaches are designed to be efficient as the node tests are low-cost and only m tests-per-tree across all L trees are needed to classify a feature (in this example, m=10 and L=90). In the case of the BWRTs, an initial tree bag of 1000 is used and the best 90 are selected from this bag.

During testing, for a given 640×480 image every other pixel is classified using the descriptor/classifier combinations. This amounts to 76,800 descriptor extractions and classifications per-frame for each algorithm. A constant-size window is used for each descriptor (21 pixel diameter, empirically determined) for all descriptor types. The average run-time per-frame is analyzed and the results are shown in the third column of TABLE 2 in msecs/frame. The higher-dimensional feature vectors required more time, especially in the case of the SVMs. Therefore, SIFT (d=128) had the largest run-time and HoG (d=45) had the smallest. The run-time for the RT and BWRT (d=66) cases should be very close as they are equivalent in terms of behavior, only differing in the values for the weights.

TABLE 2

| Descriptor | Classifier | Unmasked | Masked |
|---|---|---|---|
| Covar | SVM | 60185.4 | 4431.18 |
| | RT | 8672.4 | 1171.01 |
| | BWRT | 8685.57 | 1086.8 |
| SIFT | SVM | 204696 | 13704.8 |
| | RT | 11914.3 | 915.163 |
| | BWRI | 12325.9 | 990.732 |
| HoG | SVM | 55634.6 | 4216.58 |
| | RT | 5231.53 | 551.321 |
| | BWRT | 5388.07 | 557.324 |

The fastest algorithm was HoG/RT and HoG/BWRT, with the smallest complexity. An increase in speed can be applied to all cases if an initial mask prior were present, which would limit which pixels to analyze in the image (as applied above). The classifications can be confined to pixels only on the metal tip of the tool (as discussed above). The runtime results (including the time to compute the masks) are shown in the fourth column of TABLE 2, which shows a significant reduction in processing. This gets closer to a real-time solution, where, for example, the Covar/BWRT approach is reduced to a little over 1 sec/frame. Finally, the percent decrease in run-time from the SVM case to the RT/BWRT cases is analyzed for each descriptor. With a slight reduction in accuracy performance, this showed a reduction of up to 80% using Covar, and 90% and 94% using the HoG and SIFT descriptors, respectively. These are not trivial speed-ups, and should be considered in the choice of the feature detection algorithm.

Although some feature types may not always be detected, a minimum of 3 are required on a given frame to recover the articulated pose (because the algorithm described above fuses kinematics with vision), and so across the 7 chosen landmarks, the percent correct achieved is sufficient for longterm tracking. Features with low probabilities are rejected based on the confidence. When considering tracking 2 tools simultaneously, kinematics can be used as a prior on geometric constraints to assign features to the most likely tool pairing.

It is understood that the subject matter described herein is not limited to particular embodiments described, as such may, of course, vary. For example, various image segmentation methods can be used in accordance with the present subject matter including thresholding, clustering, graph cut algorithms, edge detection, Gaussian mixture models, and other suitable image segmentation methods known in the art. Various descriptors can also be used in accordance with the present subject matter including covariance descriptors, Scale Invariant Feature Transform (SIFT) descriptors, Histogram-of-Orientation Gradients (HoG) descriptors, Binary Robust Independent Elementary Features (BRIEF) descriptors, and other suitable descriptors known in the art. Various classifiers can also be used in accordance with the present subject matter including randomized tree classifiers, Support Vector Machines (SVM), AdaBoost, and other suitable classifiers known in the art. Accordingly, nothing contained in the Abstract or the Summary should be understood as limiting the scope of the disclosure. It is also understood that the terminology used herein is for the purpose of describing particular embodiments only, and is not intended to be limiting. Where a range of values is provided, it is understood that each intervening value between the upper and lower limit of that range and any other stated or intervening value in that stated range, is encompassed within the disclosed subject matter.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

As used herein and in the appended claims, the singular forms “a,” “an,” and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosed subject matter. Various modifications can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A robotic surgical tool tracking method, comprising:
generating a descriptor of a region of an input image;
applying a trained classifier to the descriptor to generate an output indicative of whether a feature of a surgical tool is present in the region;
determining the location of the feature of the surgical tool based on the output of the trained classifier.

2. The method of claim 1, wherein the descriptor is selected from the group consisting of a covariance descriptor, a scale invariant feature transform descriptor, a histogram-of-orientation gradients descriptor, and a binary robust independent elementary features descriptor.

3. The method of claim 1, wherein the trained classifier is selected from the group consisting of a randomized tree classifier, a support vector machine classifier, and an AdaBoost classifier.

4. The method of claim 1, wherein the region is selected from within a predetermined area of the input image.

5. The method of claim 1, wherein the region is selected from within a mask area indicative of the portion of the input image that corresponds to a tip portion of the surgical tool.

6. The method of claim 1, wherein the input image contains a plurality of surgical tools, the method further comprising:
determining to which of the plurality of surgical tools the feature corresponds.

7. The method of claim 5, further comprising:
generating the mask area by applying a Gaussian mixture model.

8. The method of claim 5, further comprising:
generating the mask area by image segmentation by color clustering.

9. The method of claim 5, further comprising:
generating the mask area by image segmentation by thresholding.

10. The method of claim 5, further comprising:
generating the mask area by image segmentation by application of a graph cut algorithm.

11. The method of claim 2, wherein the descriptor is a covariance descriptor.

12. The method of claim 11, wherein the covariance descriptor comprises an x coordinate, a y coordinate, a hue, a saturation, a color value, a first order image gradient, a second order image gradient, a gradient magnitude, and a gradient orientation.

13. The method of claim 1, wherein the classifier is a randomized tree classifier.

14. The method of claim 13, wherein the randomized tree classifier additionally comprises weights associated with each tree and applying the classifier comprises applying the weights associated with each tree to the outputs of each tree.

15. A non-transient computer readable medium for use with a robotic surgical tool tracking system, comprising:
instructions for generating a descriptor of a region of an input image;
instructions for applying a trained classifier to the descriptor to generate an output indicative of whether a feature of a surgical tool is present in the region;
instructions for determining the location of the feature of the surgical tool based on the output of the trained classifier.

16. The non-transient computer readable medium of claim 15, wherein the descriptor is selected from the group consisting of a covariance descriptor, a scale invariant feature transform descriptor, a histogram-of-orientation gradients descriptor, and a binary robust independent elementary features descriptor.

17. The non-transient computer readable medium of claim 15, wherein the trained classifier is selected from the group consisting of a randomized tree classifier, a support vector machine classifier, and an AdaBoost classifier.
